(19) United States
(12) Patent Application Publication
(10) Pub. No.: US 2008/0200818 A1
(43) Pub. Date: Aug. 21, 2008

(54) Surface measurement apparatus and method using parallax views

(75) Inventors: Scott Determan, North Andover, MA (US); Peter Miller, Cambridge, MA (US)

Correspondence Address: Cohen Pontani Lieberman & Pavane LLP, Suite 1210, 551 Fifth Avenue, New York, NY, 10176, US

(73) Assignee: Cambridge Research & Instrumentation, Inc.

(21) Appl. No.: 12/005,474

(22) Filed: Dec. 27, 2007

Related U.S. Application Data
(60) Provisional application No. 60/877,361, filed on Dec. 27, 2006.

(51) Int. Cl. A61B 6/04

(52) U.S. Cl. 600/476; 600/407

(57) ABSTRACT

The invention provides for surface mapping of in-vivo imaging subjects using a single camera having a lens which is not telecentric in object space, and a moveable stage on which a subject animal for in-vivo imaging is placed. Images are taken and the stage is moved by known amounts, and the height of individual features on the subject is determined through analysis of how much the feature shifts in the image, given the known stage displacement and lens placement. A mesh or other surface can be constructed from individual features, to provide a map of the subject. Alternatively, two cameras are used in a calibrated stereo viewing arrangement. Resolution of 0.5 mm or better can be attained for mice and similarly sized subjects.

### RELATED APPLICATIONS

This application claims priority from U.S. Provisional Patent Application Ser. No. 60/877,361 which was filed on Dec. 27, 2006.

### BACKGROUND OF THE INVENTION

In-vivo imaging systems are commercially available for imaging small animals such as mice, such as the Maestro system from CRI Inc., (Woburn Mass.), and the IVIS system from Xenogen (Hopkinton, Mass.).

Motorized sample stages are widely used in optical imaging equipment, to permit loading multiple samples, or to permit selection of a sub-region of the sample for closer examination or measurement.

Parallax rangefinders are an optical arrangement for gauging distance to an object. One implementation involves a partial mirror to superimpose two views of an object, which come from two separate optical trains having distinct entrance pupils offset by some amount δx. The views are made to align by adjusting a calibrated mirror or prism that deviates one or both beams by a known amount. From the amount of deviation and the offset amount δx, the distance to the object is determined using analytical geometry.

Design of lenses and lens assemblies is discussed in a variety of textbooks such as Modern Optical Engineering, Warren J. Smith, McGraw-Hill, 3^{rd} Edition (2000). This describes the primary aspects of a lens assembly, including entrance and exit pupils and principal plane locations, as well as the factors determining them. These may be calculated using ray-tracing programs such as Zemax from Zemax Development Corp. (Bellevue, Wash.).

The benefits of mapping the surface contours of objects to be studied by optical imaging, have been recognized in the field of in-vivo imaging. See for example, US Patent Published Application 20060268153 to Rice et. al. which describe use of structured light and photographic views toward this end.

Use of structured light methods to perform surface mapping can be costly since specialized illumination optics are required.

It is desirable to provide for surface mapping that uses elements already present in an in-vivo imaging system, with minimal change or addition, which eliminates the need for specialized optics, or specialized illumination systems. It is further desirable that the apparatus and method for surface mapping be simple and give accurate results.

### SUMMARY OF THE INVENTION

At the core of the invention is the recognition that parallax can be used as a means of sample contour mapping for in-vivo imaging, to obviate or augment structured light or other complex arrangements. For example, a motorized stage can be used to move the subject by known amounts while it is at least partially within the field of view of the imaging system. Images are taken with the subject in each stage position. By analyzing the location of individual features on the subject after the stage is moved around to each position, it is possible to determine the height above the stage surface for each feature. The process is analogous to visual depth perception. The invention provides for taking images from different known viewpoints relative to the subject, like the view from two eyes; it then triangulates to determine the position of a plurality of points on the sample surface, to determine sample contour.

According to an embodiment of the present invention, image-processing methods are used to locate recognizable features at or near the surface of the subject, such as sebum, hair follicles, eyes, moles, markings, pores, or the like. These must be located in each image and correspondence made between features in each of the multiple views. These features are distributed across the surface of the subject, and form the reference points for depth measurements. A mesh or surface is constructed from such features, and this mesh is an estimate of the actual subject contour.

A minimum of two views are used to form a depth estimate. More views can be taken, and this is especially useful when the imaging system is configured to run in close-up mode, with a relatively small field-of-view. This yields best depth resolution, though it may be necessary to take three or more images, in order to obtain at least two views of every portion of the subject.

The invention is normally practiced as part of the apparatus and method of operation of an in-vivo imaging system. This is especially favored when the imaging system provides the necessary imaging and stage apparatus already to perform other essential functions. However, the invention can be practiced on a separate apparatus, such as a separate imaging station on which the subject profile is obtained before or after another in-vivo imaging measurement of interest. The imaging measurement data may be combined with the contour information in order to interpret the results. For example, the in-vivo imaging apparatus and method may be used to obtain a fluorescence image or a set of multispectral fluorescence images. Or, it may be used to obtain an bioluminescent image of the subject. In either case, one obtains an image based on the location of chemical compounds within the subject, and the interaction of light with tissue as it propagates within the subject. Knowledge of the three-dimensional shape of the subject can be used as an input to models such as Monte-Carlo models or photon diffusion models, to obtain an improved estimate of the amount and location of chemical compounds within the subject.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings, wherein like reference characters denote similar elements throughout the several views:

FIG. 1 is a schematic perspective view of an in-vivo imaging instrument suitable for practicing the invention;

FIG. 2 is a flow diagram showing the steps to be performed for contour determination according to an embodiment of the present invention;

FIG. 3a is a schematic view of an optical system for use in the device of FIG. 1;

FIG. 3b is a schematic view of the optical system of FIG. 3a in which the stage is offset;

FIG. 4 is a view of a ‘virtual’ optical device combining the positions of FIGS. 3a and 3b;

FIG. 5 is a geometrical diagram of a bundle of light rays passing from a feature on the surface of a subject through an objective lens of the optical system of FIG. 3a;

FIG. 6 is an x-z cross-section view diagram of a measurement in accordance with the invention;

FIG. 7 is a diagram illustrating the geometry used to analyze the sensitivity of the present invention when determining z for measurements of contour; and

FIG. 8 is an image of mouse showing the sebum features that are visible as bright spots on the mouse skin.

### DETAILED DESCRIPTION OF THE PRESENTLY PREFERRED EMBODIMENTS

In this discussion, technical terms have their normal meanings unless stated otherwise. “Subject” refers to an intact animal subject, including without limitation a mouse, rat, human, cat, dog, monkey, zebrafish, or any other creature for which it is desired to learn its surface contours. “Object” refers to a physical object, such as a subject, whereas “image” refers to an image formed by optics of some kind.

“Lens” means a lens or lens assembly. “Light” means light of any type whatsoever, including ultraviolet, visible, and infrared light. “Stage” means an apparatus for holding or supporting a subject. “Height” means a specified distance above the stage. Unless stated otherwise, the coordinate system is defined such that height, corresponding to the Z-axis, is perpendicular to the stage surface; and the X-Y plane is an imaginary plane at the height of the stage or a portion thereof.

The invention is best explained by relating specific embodiments. It will be understood, however, that this is done for ease and clarity of illustration, and that the invention can be practiced more broadly, or with different apparatus, without deviating from its spirit.

The invention has as its aim the determination of the surface contours of a subject. Commonly, this is a subject intended for measurement in an in-vivo imaging experiment such as a fluorescence or bioluminescence imaging experiment. However, the invention can be used to measure the surface contours of subjects for other measurements as well. It is a further goal of the invention to avoid the need for structured light, or to augment it rather than to rely upon it. That is, the invention may be practiced without need for structured light apparatus, though it does not interfere with such illumination if that is desirable for other reasons.

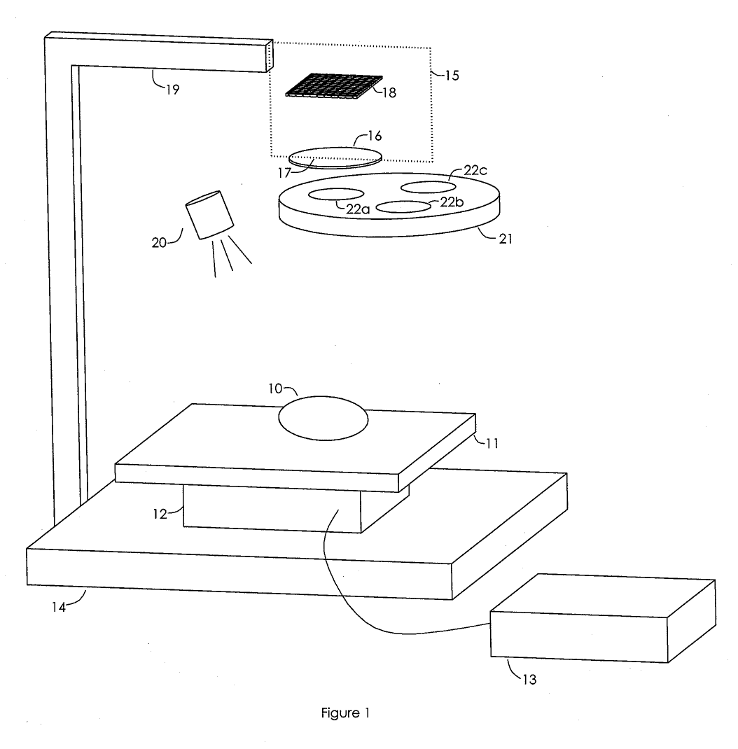

FIG. 1 is a schematic representation of an in-vivo imaging instrument suitable for practicing the invention. A subject 10 is placed on a stage 11 which is moveable by motion control elements 12 controlled by computer 13. The control elements 12 are connected between the stage 11 and a chassis 14. An imaging system 15 includes an objective lens 16 having an entrance pupil 17 and a sensor 18, and is connected to the chassis 14 by support member 19. Illumination source 20 illuminates the subject. Optional filter wheel 21 in front of the objective lens 16 selects filter 22a, 22b, or 22c to define a wavelength band or bands for the imaging measurement. The subject may optionally be supported or surrounded by apparatus to provide a controlled temperature environment, or to restrain it against unwanted movement, or to deliver anesthesia, or combinations of these.

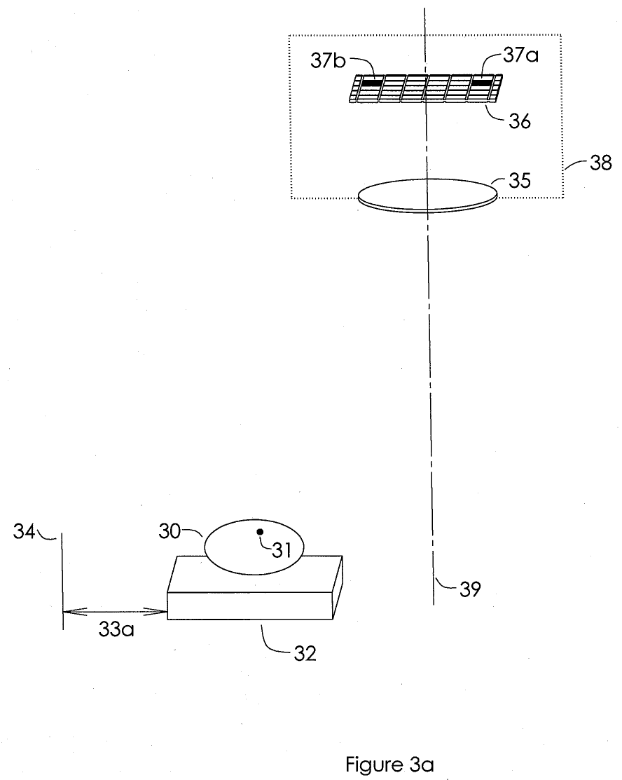

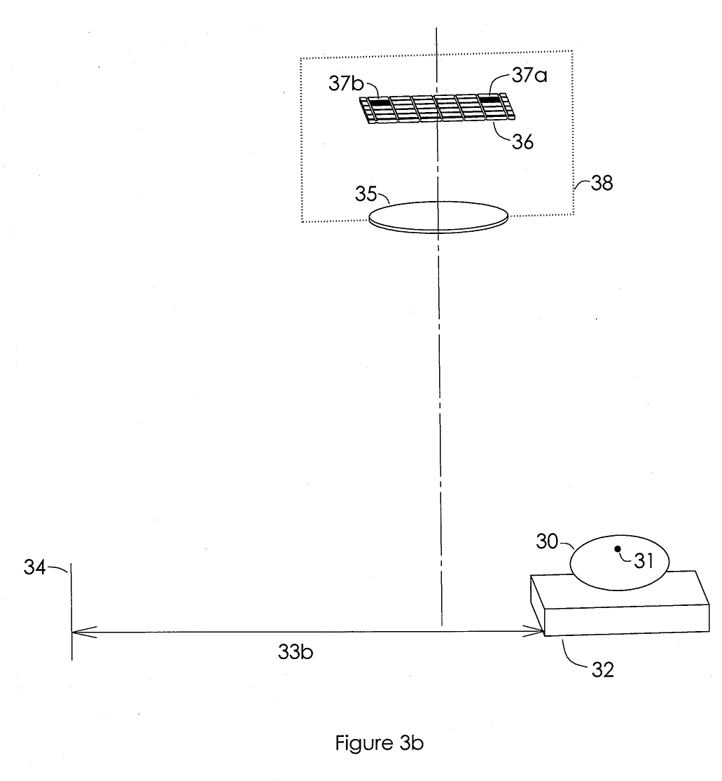

FIG. 3a shows a schematic diagram of an optical system which may be used in the device of FIG. 1. FIG. 3a shows a subject 30 with feature 31 on stage 32 in a first position that is offset by distance 33a from a reference point 34. Objective lens 35 and imaging detector 36 having pixels 37a and 37b form an imaging system 38. An image of feature 31 is formed at pixel 37a. The optical axis is indicated by 39.

FIG. 3b shows a schematic diagram of the optical system of FIG. 3a, except the stage 32 is in a second position that is offset by distance 33b from a reference point 34, and an image of feature 31 is formed at pixel 37b.

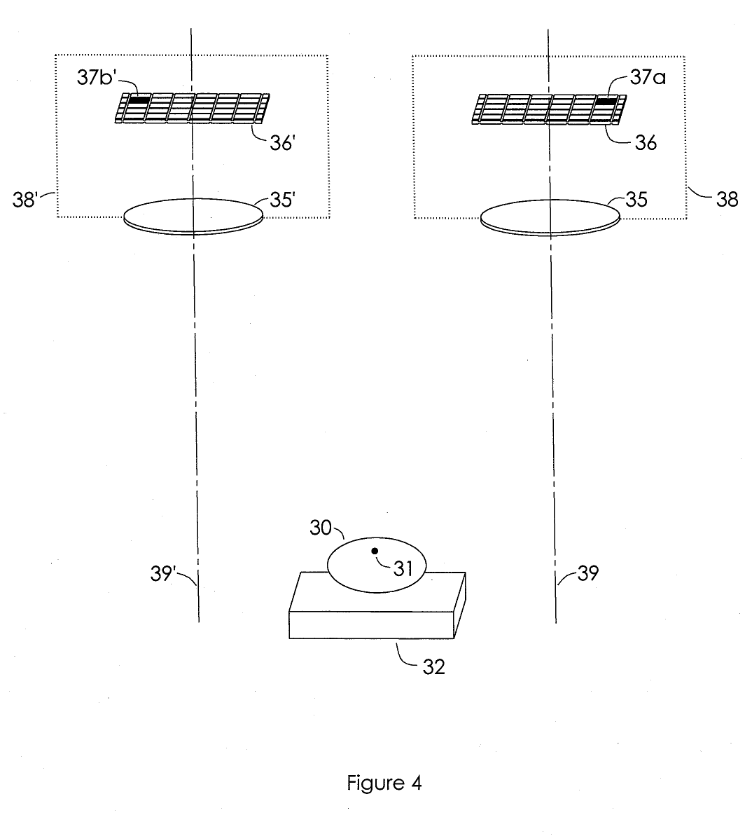

FIG. 4 shows a diagram of a ‘virtual’ apparatus which is equivalent to that produced by the apparatus of FIGS. 3a and 3b. It depicts imaging system 38 in the same position relative to the subject that was in effect in FIG. 3a, and imaging system 38′ in the same position relative to the subject that was in effect in FIG. 3b. This illustrates how the invention achieves depth perception and thus contour mapping.

A first image of the subject is recorded by the imaging system with the stage in a first position, and then the stage is moved by a known amount δx and a second image is recorded. The lens is focused to achieve a sharp image for objects that are coplanar with that portion of the subject being imaged, or nearly so, and is the same for both images.

Since the goal is to map the contour of the subject, it will be understood that the subject spans a range of heights. Similarly, the lens has a finite range over which objects can be clearly distinguished, which is denoted its depth-of-field. This depends on the aperture and magnification, as is known in the optical art. It is often desirable to select an optical system for which the depth-of-field is sufficient to resolve features on the subject surface, over the range of heights for which one wishes to obtain contour maps. Since the features are themselves typically several pixels in extent, or larger, it is not necessary that the depth-of-field be great enough to provide pixel-limited sharpness. It is only necessary that the feature be detected and its location be determined, which requires a less critical degree of sharpness.

If it is impossible to attain this range of depth-of-focus, one can practice the invention by first mapping out the contour elements that fall within a first sub-range, then refocusing to optimally work in a second sub-range, and mapping the contour elements in that range, and so on, until the full contour has been obtained. Alternatively, the relative position of the subject and the imaging system can be changed, for example by moving the height of the stage by a known amount, which must be accommodated for in the subsequent calculations.

For simplicity, the lens is treated in this discussion as if it were a perfect, thin lens element that obeys the paraxial lens approximation. This idealized lens assumption makes it easy to draw and understand optical diagrams, but such lenses do not exist in practice. One preferably will use a highly corrected lens such as the Micro-Nikkor AF60 D F/2.8 lens from Nikon (Melville, N.Y.), which has excellent imaging properties and yields nearly-ideal imaging performance.

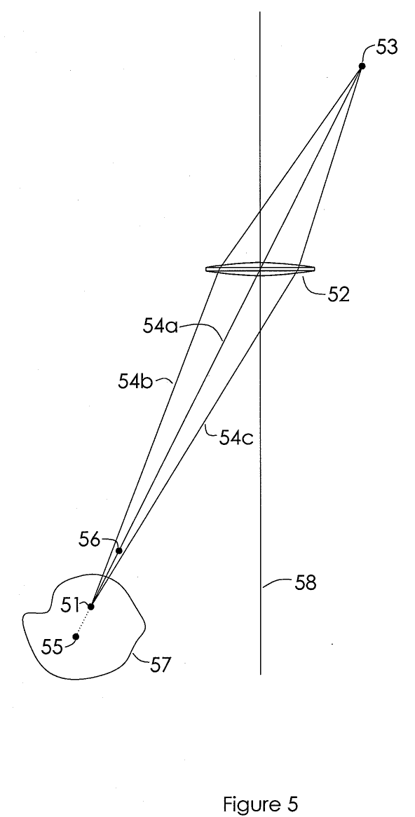

FIG. 5 shows a geometrical diagram of a bundle of light rays 54a-54c, passing from a feature 51 on the surface of a subject 57, through objective lens 52, to form an image at point 53 on a sensor. Ray 54a is the chief ray of the bundle and passes through the center of objective 52. Points 55 and 56 also lie along the direction of chief ray 54a but do not correspond to points on the surface of subject 57; point 55 lies within the subject and point 56 is a point in free space surrounding the subject. The optical axis of the system is indicated by 58.

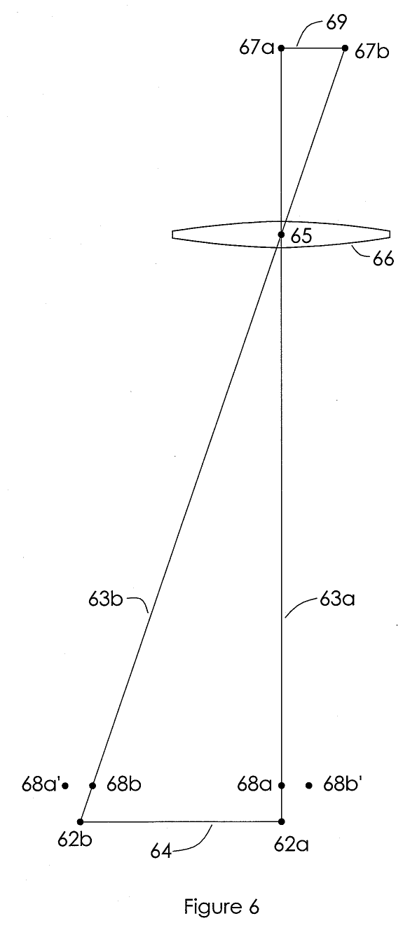

FIG. 6 shows an x-z cross-section view diagram of a measurement in accordance with the invention. It depicts feature 61 in first location 62a and in second location 62b, separated by displacement 64 in the x direction. Location 65 corresponds to the position of the entrance pupil of an objective lens 66, which is an idealized paraxial thin lens. Lens 66 forms images of location 62a and 62b at positions 67a and 67b, respectively, separated by displacement 69 in the x direction. Light travels along chief ray 63a from point 62a to pixel 67a, and along chief ray 63b from point 62b to pixel 67b. Points 68a indicates a point adjacent to feature 61 along chief ray 63a when the subject is in the first location, and point 68a′ indicates the same point when the subject is in the second position. Point 68b indicates a point adjacent to feature 61 when the subject is in the second position, and 68b′ indicates the same point when the subject is in the first position.

The optical arrangement is diagrammed in FIG. 6. A feature that is detected at pixel 67b must lie along chief ray 63b. However, that knowledge alone is not enough to localize the feature in 3D space, since there are an infinite number of [x, z] points that lie along that line. For example, one cannot distinguish whether the point corresponds to location 62b or 68b. Similarly, a feature that is detected at pixel 67a must lie along chief ray 63a, but that measurement is unable to discern between a point at location 62a and 68a. However, when the measurements are combined, there is only one z value that is consistent with both observations, and this must be the location of the actual subject feature. From the z-value, the x-value is then determined by trigonometry.

Several considerations are important in order to get good results. First, note that location 65 in FIG. 6 corresponds to the center of the entrance pupil of the objective lens. The distance between this point and the subject should be kept low in order that the angles in the above diagram are large enough to measure with adequate resolution. FIG. 6 shows an idealized system, and the pupil 65 is shown within lens 66, but when real lens system are considered, the pupil 65 may not lie within the lens at all; indeed, for some lens designs, the pupil can appear to be infinitely far away. Such a lens system, which is said to be telecentric in object space, is sometimes favored in optical instrumentation because it has certain desirable imaging properties. However, when the stage is moved, such a lens produces a shift in the image that is independent of the height (or z-value) of the feature being imaged; thus they are unsuitable for this embodiment.

This can be seen another way, as well. For such a lens, location 65 is located at a location with large, almost infinite z. Thus the chief rays 63a and 63b are both essentially vertical, and it will be impossible to distinguish between the actual feature 62a from others such as 68a having a different z-value.

The minimum resolvable depth difference is an important figure-of-merit for a contour measurement system. Here we shall provide an estimate of this quantity for the invention in the case where the limiting angular resolution is that of a single pixel in the sensor. In this estimate, we use the thin-lens paraxial approximation where the principal planes and pupils are coincident with the lens itself and the lens imaging properties are perfect. The latter condition can typically be attained with modern lens assemblies. To apply the results of this analysis to a real system, one may determine the actual locations of the pupils and principal planes of a lens using ray tracing programs or measurements. So these simplifications do not materially alter the result, compared to what can be attained in practice, or when a more detailed analysis is performed.

Modern scientific imaging sensors typically offer megapixel resolution, or higher. For example the Sony ICX-285 sensor has 1024×1392 resolution, meaning it is possible to discern 1000 spots or more, in any direction. The QImaging Retiga EXi from QImaging (Burnaby, British Columbia, Canada) is an example of a camera using this sensor. Similarly, the Texas Instruments Impactron EMCCD offers 1004×1002 pixel resolution, and is used in the iXon EM DV885-LC camera from Andor Technology (Belfast, Northern Ireland). Both of these cameras offer very high sensitivity and are suitable for scientific grade imaging.

It is often beneficial to practice the present invention together with, or as part of, a high-performance measurement system such as an in-vivo fluorescence or luminescence measurement system. In such cases, an imaging sensor may already be present for purposes of making other measurements. When this is the case, it can be beneficial to use that existing imaging sensor for practicing this invention. However, this is not essential, and it is possible to use a separate imaging sensor, or a lower-performance imaging sensor, for practicing this invention. All that is important is that the sensor have sufficient size and resolution to provide the necessary images. It can be possible to use CMOS imaging sensors or other low-cost imaging sensors to practice the invention in cases where the sensor is not used for other measurements or subjected to other constraints.

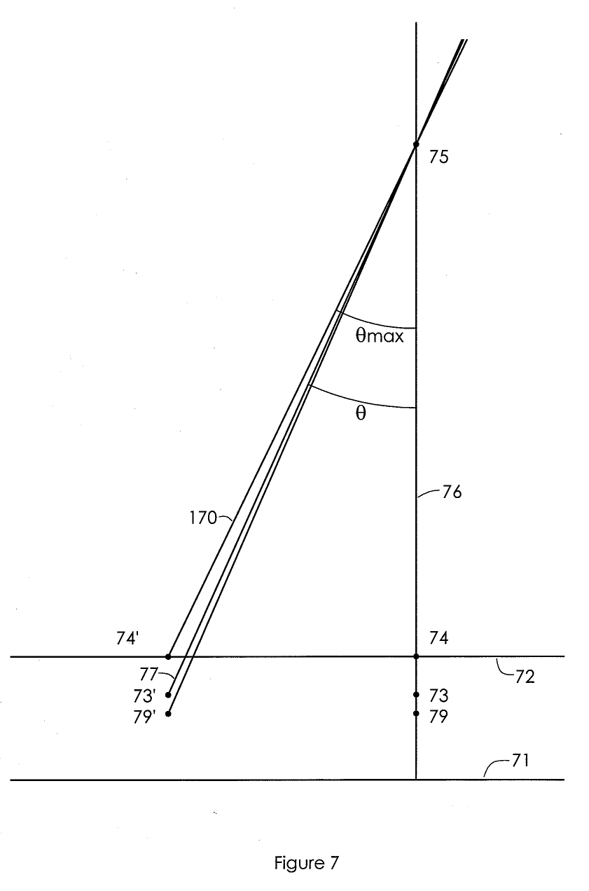

Consider the case where a subject, or a patch of the subject, would occupy the center of the first image, and the translation δx is chosen so that same subject or patch would occupy the left-most point of the second image if it had a height Zₘ which is higher than any portion of the actual subject. This is diagrammed in FIG. 7, which shows the geometry used to analyze the sensitivity of the invention when determining z for measurements of contour. FIG. 7 shows line 71 representing the stage, which defines the coordinate z=0; and line 72 representing the maximum possible subject height based on some a priori knowledge of the subject range of variability. Point 73 represents the location of a subject feature when the stage is in a first position, and point 73′ represents the location of the subject feature when the stage is in a second position. Point 74 represents a point directly above 73 which lies on line 72. Point 74′ represents the same point when the stage is in the second position, when it would fall on the margin of the image, shown by line 170. In the first stage position, points 73 and 74 lie directly below the center of the lens entrance pupil 75, along the optical axis 76 of the system. Line 77 represents the chief ray for light traveling from 73 to the lens entrance pupil, and line 78 represents the chief ray for a point 79′ which lies a distance δh below point 73′, whose height differs just enough to be detected as distinct from 73′. In the second image, point 73′ lies at an angle θ away from the optical axis, which is less than θₘₐₓ because the point 73 lies below the predetermined maximum sample height, indicated by line 72. Angle θₘₐₓ indicates the half-angle viewed by the imaging system, and 0 indicates the angle between chief ray 77 and the optical axis.

One can determine the angular resolving power of the imaging sensor, by which we mean the smallest angular shift δθ that it can resolve in the chief ray coming from the subject. In the small-angle approximation, this is seen to be

δθ=θₘₐₓ/(N/2)=2θₘₐₓ/N [1]

where N is the imaging sensor resolution along the x-axis. Again, we presume a resolution of one pixel though in practice finer resolution can sometimes be attained by use of correlation techniques. If one denotes the distance between the lens pupil 75 and the feature location 73′ as R, then by trigonometry we may write

67 z=Rδθ/sin θ [2]

where δz is the smallest resolvable height difference in the subject, such as that between 73′ and 79′.

One may consider some practical cases of interest. Suppose one uses a 50 mm lens and operates it at a 1:1 conjugate ratio, to produce a 1× image of the subject. This is imaged on a Kodak KAF-4202 imaging sensor (Kodak Image Sensor Solutions, Rochester N.Y.). This yields an 18 mm square image of the subject, with 2000×2000 pixel resolution, so N=2000. The subject is a mouse, which has a maximum height of 20 mm, and the lens is focused at the midpoint of the mouse.

For the simple lens, the working distance from pupil to subject is 2 F, or 100 mm, and θₘₐₓ is given by

θₘₐₓ=arctan(9 mm/90 mm)=0.09966 [3]

R is 100.4 mm, by Pythagorean equation using the working distance of 100 mm and the x-displacement of 9 mm. We further calculate

θ=arctan(9 mm/100 mm)=0.08975 [4]

δθ=2θₘₐₓ/N=0.00009966 [5]

From these, the resolution for the height measurement δz is given by equation [2] as

δz=Rδθ/sin θ=0.11 mm [6]

Thus, in two measurements one has attained excellent depth resolution over a patch spanning 9 mm in x dimension, and 18 mm in the y dimension.

The same system can be operated at a 2:1 conjugate ratio, for which the working distance between entrance pupil and subject is 150 mm, and the imager records a 36 mm square region of the subject. The maximum sample height location is 140 mm.

If larger field of view is desired, the same system can be operated at 4:1 conjugate ratio, to view a 72×72 mm sample region. It may be analyzed using the same equations and methodology.

We tabulate the principal quantities for the three cases as follows:

TABLE 1
Depth resolution for sample instrument used at various magnifications.

| | Working | Sample | | | | | |
|---|---|---|---|---|---|---|---|
| Mag | distance | region | θₘₐₓ | δθ | θ | R | δz |
| 1:1 | 100 mm | 9 mm × 18 mm | 0.9966 | 0.0000997 | 0.0897 | 100 mm | 0.11 mm |
| 2:1 | 150 mm | 18 mm × 36 mm | 0.1279 | 0.0001279 | 0.1194 | 151 mm | 0.16 mm |
| 4:1 | 250 mm | 72 mm × 72 mm | 0.1489 | 0.0001489 | 0.1430 | 253 mm | 0.26 mm |

In other instruments, the sensor size and pixel resolution may be different, but the principle of operation, and method of estimating the degree of depth resolution that can be attained, is similar.

The example above considers the case where the stage is moved by an amount that produces a shift one-half the size of the image between the two images. However, other movements may be used according to the need at hand. What is important is that a given feature be visible in at least two images, taken with the imaging system at different known viewpoints relative to the subject.

Nor do all features need to be visible in the same pair of images. For example, in one aspect of the invention, sub-regions of the subject are imaged and three or more images are taken while the subject is moved to the right. A given subregion A appears in the left-hand portion of the first image while the right-hand portion of the first image is empty. In the second image, sub-region A appears in the right-hand portion, while sub-region B appears in the left-portion of that image.

Note that upon acquisition of the second image, one has enough information to produce a contour map of the first sub-region but not the second sub-region B.

In the third image, sub-region B appears in the right-hand portion of the image. It is possible at this point to produce a contour map of both sub-regions A and B. In some cases, this pattern of movement and image-taking is continued for additional images, while other sub-regions are measured; in other cases, only two sub-regions are to be measured, and a total of only three images are required.

The overall process consists of the measurements just described, along with a process for data analysis. In any practical system, the overall contour measurement is done in an automated manner, using computer analysis of the various images. So, in addition to the geometrical calculations described above, it is necessary to perform automatic detection of the features in each of successive images; automated assignment of a location to each feature; automatic correlation of which feature in each image corresponds to the same feature in other images; and construction of the contour from the mesh of features, once the height is determined for each one.

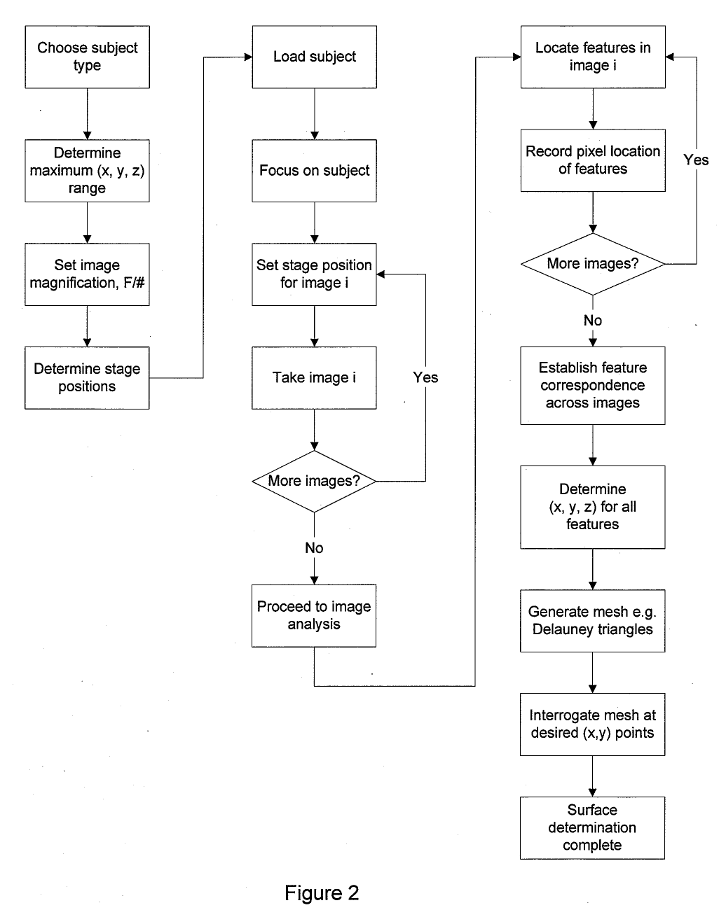

The overall measurement process is shown in FIG. 2. A subject is loaded, the stage location is chosen, the stage is set to that position, an image is taken, and the process is repeated according to the flow-chart logic, until all subject regions of interest have been imaged in at least two different stage positions. Then, subject features are identified in each image, and image coordinates assigned to each feature. The correspondence between features in different images is determined. From the position in each of two images, and the known viewpoint displacement, the feature height is determined. The x and y coordinate of each feature is also determined, relative to a coordinate system. Each feature is added to a list of features and 3-dimensional locations to form a list of features. From the feature list, a contour surface is generated.

The feature detection can be done using image processing techniques such as thresholding based on intensity, contrast, and size. Other processing steps such as template matching, texture analysis, and color analysis, can be applied as well. Feature location can be done using a center-of-mass calculation for the pixels within a feature. Correlation can be done by choosing the nearest feature in corresponding images, after applying the transform based on the known stage translation for a nominal height, such as the mean subject height. The contour can be constructed by a variety of methods, such as constructing a list of all features; generating an x-y grid and interrogating the list for the nearby points from which an interpolated value for z is developed at the grid location. The method known as Delaunay triangulation can be used to construct a set of triangles from a set of feature locations in space, within which triangles one may interpolate a surface.

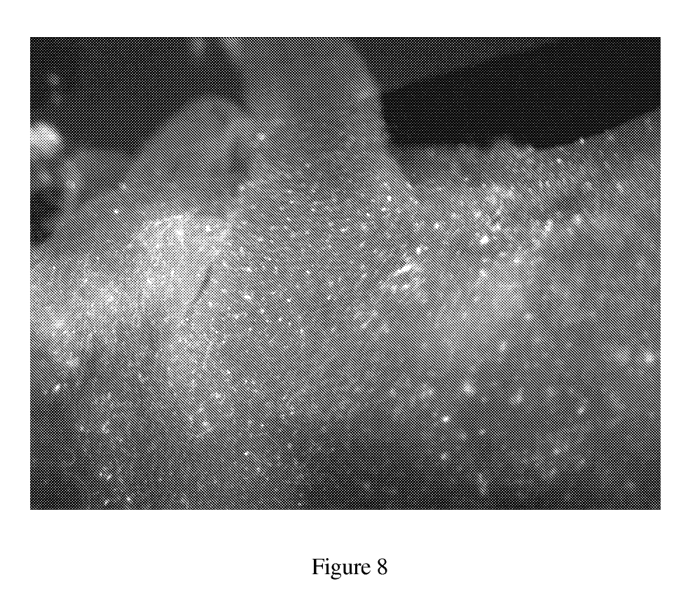

The images used for this process can be any type which reveal features of adequate contrast for the measurement of feature location. For example, fluorescent images can be used where that reveals features. FIG. 8 shows a fluorescent image of a nude mouse subject which was illuminated with excitation light in the 480 nm range, and viewed through a long-pass filter with 520 nm cut-in. The mouse exhibits generalized autofluorescence over most of its surface, with punctuate bright regions corresponding to sebum on the subject. These sebum features would be suitable features for use in this invention.

However, the invention is not limited to use in fluorescent imaging modes. Ordinary reflected light imaging can also be used if that is preferred, and if it yields features that can be detected for a given subject. Also, spectral imaging may be employed, and individual component planes associated with a known spectrum may be used for feature detection; this is valuable for enhancing feature contrast when the features are associated with a particular spectral shape.

Indeed, any imaging mode may be used that provides feature location data, and the choice can be made based on factors such as what imaging modes are available for a given set of apparatus; what types of subjects are to be viewed and what features are present; and speed of image acquisition.

In some cases it may be preferred to provide two cameras rather than one camera with a moving stage. This can be done, and the same general mathematical approach employed, though it may be necessary to calibrate the two cameras to take account of any factors such as slight differences in the lens systems, optical axis pointing, and the like; which arise because of implementing the invention this way rather than as described above.

In some cases it may be preferred to provide a separate station rather than to make this part of a larger instrument system. This may be done because the imaging sensor in the larger instrument system is not suitable for the contour measurements; or because it is desired to provide the contour measurement as an accessory to an existing system; or to provide increased temporal throughput for the workstation; or for a variety of reasons. In any case, it is possible to perform the contour measurement before or after another measurement of interest; and then to combine the contour information with the results of the other measurement to arrive at a more complete understanding of the subject.

While it is expected that the invention is normally practiced on subjects which are anaesthetized or otherwise immobilized, the subject may still exhibit slight movement due to respiration and circulation functions. If it is desired, one may synchronize the image acquisition with subject breathing or heartbeat to reduce the effect of these factors on the measurement. Alternatively, several images can be taken which span the period of the movement, from which an improved estimate of position can be obtained by taking a mean value, or in the case of a repetitive movement, interpreting the images to determine which position corresponds to a specific state in the movement cycle.

Thus, while the invention has been described by use of specific, other embodiments can be employed without deviating from the invention. For example, one may employ a wide range of imaging sensors and lenses, consistent with the need to yield a suitable image of the subject. Similarly, any stage may be used that can move the subject over the required range of positions. Turning to the data analysis and derivation of a contour surface, there are many ways to exploit the core depth perception approach that the invention provides, and the methods shown herein should be considered a non-limiting guide to how this can be accomplished. Alternative approaches can be used, such as lookup tables, numerical calculations, and so on, provided that the result is that a height estimate is determined from the apparent position of a feature in two images taken from different viewpoints relative to the subject. Accordingly, it is understood that the scope of the invention is limited only be the attached claims, and not by the specific examples and embodiments.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed:

1. A method for determining a contour of a subject using an in-vivo measurement system having a stage supporting the subject, control elements connected to a controller, and an imaging system having an optical axis, the method comprising the steps of:
incrementally moving, by the control elements, at least one of the stage and the imaging system to a plurality of positions such that the relative movement is in a direction substantially orthogonal to the optical axis of the imaging system;
taking, by the imaging system, an image of the subject at each of the positions;
determining, by the controller, pixel locations of a plurality of features in each of the images;
establishing, by the controller, correspondence of like features between each of the images;
performing, by the controller, a height estimation algorithm for each of the plurality of features to determine the height of each of the features in a direction of the optical axis; and
constructing, by the controller, a contour from the coordinates of each of the features.

2. The method of claim 1, wherein the step of performing height estimation algorithms comprises trigonometric analysis based on the location in each of the images and relative movement between the positions.

3. The method of claim 2, wherein the individual features comprise one of sebum, hair follicles, eyes, moles, markings, and pores.

4. The method of claim 1, wherein said step of constructing comprises constructing a mesh from a network of the individual features as an estimate of an actual contour of the subject.

5. The method of claim 1, wherein the subject is a mouse or a rat.

6. The method of claim 1, wherein said step of taking images is performed using ordinary reflected ambient light.

7. The method of claim 1, wherein said step of taking images comprises taking fluorescent images.

8. The method of claim 1, further comprising the step of taking an in-vivo image of the subject for an in-vivo imaging experiment and analyzing the in-vivo image using the constructed contour.

9. The method of claim 8, wherein the in-vivo image is obtained using fluorescence or bioluminescence emitted from the subject.

10. The method of claim 9, wherein the step of taking images of the subject at each of the positions is performed using reflected light.

11. The method of claim 1, wherein each of the features is present in at least two images of the plurality of images.

12. The method of claim 1, further comprising the step of detecting, by the controller, features in the taken images using image processing techniques.

13. The method of claim 1, wherein said step of determining coordinate comprises determining Cartesian coordinates having a z-axis along the optical axis of the imaging system.

14. An in-vivo imaging system, comprising:
a stage for supporting a subject;
an imaging system having an optical axis;
control elements connected to a controller, the control elements capable of moving at least one of the stage and imaging system in a direction substantially orthogonal to the optical axis of the imaging system, the controller storing an executable program for determining a contour, the program comprising the executable steps of:
incrementally moving, by the control elements, at least one of the stage and the imaging system to a plurality of positions such that the relative movement is in a direction substantially orthogonal to the optical axis of the imaging system;
taking, by the imaging system, an image of the subject at each of the positions;
determining, by the controller, pixel locations of a plurality of features in each of the images;
establishing, by the controller, correspondence of like features between each of the images;
performing, by the controller, a height estimation algorithm for each of the plurality of features to determine the height of each of the features in a direction of the optical axis; and
constructing, by the controller, a contour from the coordinates of each of the features.

15. The system of claim 14, wherein the program further comprises the executable step of taking an in-vivo image of the subject for an in-vivo imaging experiment and analyzing the in-vivo image using the constructed contour.

16. The system of claim 15, wherein the in-vivo image is obtained using fluorescence or bioluminescence emitted from the subject.

17. The system of claim 16, wherein the step of taking images of the subject at each of the positions is performed using reflected ambient light.[b2]
